(19) United States
(12) Patent Application Publication
(10) Pub. No.: US 2023/0359513 A1
(43) Pub. Date: Nov. 9, 2023

(54) ORCHESTRATION SYSTEM AND METHOD TO PROVIDE CLIENT INDEPENDENT API INTEGRATION MODEL

(71) Applicant: Marsh (USA) Inc., New York, NY (US)

(72) Inventors: Aparna Kumar Tummala, Peoria, AZ (US); Sandeep Kunjupillai Asokan, Phoenix, AZ (US)

(73) Assignee: Marsh (USA) Inc., New York, NY (US)

(21) Appl. No.: 17/749,541

(22) Filed: May 20, 2022

Related U.S. Application Data
(63) Continuation of application No. 17/736,264, filed on May 4, 2022, now abandoned.

(51) Int. Cl. G06F 9/54; G16H 80/00

(52) U.S. Cl. CPC G06F 9/541; G06F 9/547; G16H 80/00

(57) ABSTRACT

An application programming interface (API) orchestration system and method are disclosed to provide communication between clients and providers, all communicating in different formats and protocols and having different security requirements. It employs an orchestration module that has a data model creator which aggregates data fragments of the clients into a standard canonical data model. It also includes universal API gateways to connect to a plurality of adapters, each designed to be compatible with a specific provider. Each adapter includes a mapper that converts input data formats to that of a specific provider. A parameter device that for each workflow step appends parameters to messages between the client and the provider, and a protocol device, to reproduce the communication protocol of its associated provider. Each adapter has a universal API gateway that is compatible with that of the orchestration module, allowing other adapters to be added as plugin modules.

### CROSS-REFERENCES TO RELATED APPLICATION DATA

The present application is a continuation of U.S. patent application Ser. No. 17/736,264 filed on May 4, 2022, which is incorporated herein by reference in its entirety.

The present disclosure relates to an Application Program Interface (API) orchestration system and method to provide API integration and workflow management.

### BACKGROUND

It is very common to have a client connect to an online company or service. The clients may be web users, mobile users, automated systems, etc. These online connections are referred to as “end-to-end” or “E2E” connections. For example, clients may connect to service providers for various services or products, provide, retrieve or transmit requested information to the service providers, via provider systems, or pay associated fees.

### Incompatible Communication

The problems arise with these E2E connections that each online service employs a platform with different interfaces with different data formats, protocols, and security. Each will require a customized interface (translator). These will require unique client integrations and process workflows.

There is little reuse in a direct client-provider integration since direct, customized connections with the provider are built for each client.

An analogy would be when several people who speak different languages are trying to communicate with each other. The clients speak a different language than service providers via pprovider systems and cannot communicate electronically without modification of data and protocol. Manually configuring these communication connections is very time-consuming and not practical for many clients.

### Limited Dynamic System Configurability and Data Mapping

There have been translators developed that attempted to perform translations between clients systems and providers' systems.

The translators are usually based upon a predetermined workflow or fixed data model, which results in fixed data mapping. These systems will function; however, they have scalability problems and fail when providers change their data formats, protocols, or security requirements.

Manual configurations and/or programming are then required to allow the system to function again. Such systems do not provide a dynamically configurable system and/or data mapping.

### Limited Auto-Recovery from System Failures

Since these service providers may be operating continuously, they tend to have operational errors, which may cause them to reboot. Most of these systems are not very resilient and lose the data calculated to this point. Therefore, clients will have to re-start each of their processes and lose input, configurations, and other information completed until the reboot. Known systems generally are not able to retain the work completed and do not allow clients to continue their processes at the point when the system rebooted.

### SUMMARY

The present disclosure provides an application program interface (API) orchestration system that can translate using mapping and workflows, as described herein, between client systems and provider systems' data format, protocol, data collection workflow, and security. The current system also adjusts data format, has a dynamically configurable provider interaction model, has dynamic data mapping, and can automatically recover from provider system failures. Aspects of the present disclosure provide an API orchestration system and method that creates a client-independent and provider-independent API integration model. The current system also employs a data-aggregation-based integration strategy within the constraints of a canonical data model. It exhibits workflow management capabilities powered by an intelligent routing system and dynamically configurable data-driven methods. Request fulfillment routes (0-n providers), e.g., requests made to some number of providers of a product/service, are selected based on request data elements, e.g., information/data defining a request to some number of the providers of the product(s)/service(s), that are dynamically configurable. The population of data in the request determines which, if any, of the providers will be able to fullfill the request—partially or completely. Provider requests are triggered only if the minimum threshold for a provider service is met. A provider request may be retriggered in a subsequent client request if the minimum mandatory data or any other previously submitted data for the same provider request has changed.

According to one aspect of the present disclosure, the API orchestration system is disclosed that translates communications between various types of users (e.g., clients) with online providers, for example, insurance providers, online retailers, SaaS servers, and others.

Clients connect to an orchestrator module or set of orchestrator modules. The current system has a plurality of adapters that all couple to an orchestrator module through a standardized interface. Each adapter is designed to work with and connect to a specific provider.

### Orchestration Module

According to another aspect of the present disclosure, the current API orchestration system employs a client-facing orchestrator module that acts as an intermediary that provides a consistent industry-standard interface independent of provider variations, simplifying the system.

The orchestrator module employs a common standard language to communicate with the clients. In the current embodiment, the standard language chosen is an industry standard ACORD Digital language from the Association for Cooperative Operations Research and Development (ACORD), in JavaScript Object Notation (JSON), using OpenAPI and Standard Security. However, it should be appreciated that other digital languages may be used to implement the concepts described herein, and/or the chosen language may be chosen to be optimized to a particular industry, or more generally implemented languages may be used.

In the illustrative embodiments described herein, a canonical data model (CDM) is used as the data format standard for client communications. An orchestrator module according to the disclosure collects and aggregates CDM data fragments from the connected clients to assemble the aggregate CDM.

The orchestrator module employs a provider selector, which selects a services provider for each client based upon input received from the client.

The orchestrator module also includes a provider router that makes a connection between the client and the selected provider(s).

In another aspect, if the data formats, protocols, and security requirements were all built into the orchestrator module, it would result in a highly complex orchestrator module, which would be difficult to maintain or modify. Therefore, the portions unique to each provider are extracted from the orchestrator module and incorporated into an adapter specific to each provider.

The orchestrator module according to the disclosure has a plurality of universal input and output interfaces, each adapted to communicate with a connected device in a predetermined standardized data format and protocol. The protocols could be synchronous or asynchrnonous.

### Adapters

According to another aspect of the present disclosure, each adapter has a plurality of universal input and output interfaces referred to as universal API gateways (or simply “API gateways”) compatible with and connected to the universal API gateways of the orchestrator module.

These allow for universal connections, allowing for different adapters to be “plugged in” and dynamically added to the system.

The adapter includes a mapper component that maps data fields of the canonical data model and adds parameters to and from the corresponding data fields of the provider.

The protocol required by each provider is stored in a protocol device of the adapter corresponding to the provider.

A provider input/output device is coupled to the mapper and the protocol device that receives data in the data format of the provider from the mapper and communicates the data to the provider according to the protocol stored in the protocol device.

The API orchestration system according to the disclosure, in an embodiment, also includes a persistence device with a separate power supply from the remainder of the system so that it is functional in the event that the systems powering other elements of the current system fail. The persistence device acts independently from the remainder of the system. The persistence device constantly stores the current state of the orchestrator module and the adapters.

In the event that one or more of these elements fails, the persistence device will still be running and have an up-to-date version of the state of these system devices. It can then reload the failed devices with their last stored state and continue processing. This type of recovery will make these errors transparent to the clients, except for a short delay.

The API orchestration system is scalable. As providers are added, new adapters may be dynamically added as plugins to expand the system's capabilities. Adapters can also be plugged into multiple orchestration systems if needed. The API orchestration system is also flexible. The workflows can be changed to accommodate processing format changes.

The above structure and functionality result in client data flows independent of provider integration models. An API integration model is created by the client, tailored for client data flow based on a data aggregation strategy within the constraints of the canonical data model and API resources at the top level of the CDM, without a predefined data flow sequence or data fragments. The integration model dynamically supports requests at any level of the data model. Client data aggregation is done by the orchestrator and provider data mapping is done by the adapters, as described herein.

The current system employs dynamic, data-driven workflow using a canonical data model. The current API orchestration system provides complete decoupling of client and provider data. Idempotent APIs are combined with dynamic integration models. Provider request and responses are persisted with hashed identifiers guaranteeing that any change in client data results in update(s) to all impacted provider(s). Provider requests are repeated only if the request data for a provider-request has changed, otherwise the persisted result is returned. This behavior is independent of provider's idempotency capabilities and client interactions.

This has outlined, rather broadly, the features and technical advantages of the present disclosure in order that the detailed description that follows may be better understood. Additional features and advantages of the present disclosure will be described below. It should be appreciated by those skilled in the art that this present disclosure may be readily utilized as a basis for modifying or designing other systems and methods for carrying out the same purposes of the present disclosure. It should also be realized by those skilled in the art that such equivalent systems and methods do not depart from the teachings of the present disclosure as set forth in the appended claims. The novel features, which are believed to be characteristic of the present disclosure, both as to its organization and method of operation, together with further objects and advantages, will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

So that the above-recited features of the present disclosure can be understood in detail, a more particular description, briefly summarized above, may be had by reference to aspects, some of which are illustrated in the appended drawings. It is to be noted, however, that the appended drawings illustrate only certain typical aspects of this disclosure and are therefore not to be considered limiting of its scope, for the description may admit to other equally effective aspects. The same reference numbers in different drawings may identify the same or similar elements.

The features, nature, and advantages of the present disclosure will become more apparent from the detailed description set forth below when taken in conjunction with the drawings, of which:

FIG. 1 is a functional block diagrammatic overview of a prior art translation system.

FIG. 2 is a functional block diagrammatic overview of an API orchestration system according to one aspect of the present disclosure.

FIG. 3A is a partial functional block diagram of the API orchestration system showing greater detail of an orchestrator module.

FIG. 3B is a partial functional block diagram of the API orchestration system showing greater detail of an adapter.

FIG. 4 is a flowchart illustrating execution of workflow steps of one embodiment of an API orchestration system according to the present disclosure.

FIG. 5A is a flowchart illustrating the system initialization and scheduled periodic, configuration reload functions of one embodiment of an API orchestration system according to the current disclosure.

FIG. 5B is a flowchart illustrating the request/response functions of one embodiment of an API orchestrator system according to the current disclosure.

FIG. 6A shows the orchestration and adapter system initialization and periodic (scheduled) update sequence diagram illustrating orchestrator and adapters reaching out to the config provider to get their respective configurations.

FIGS. 6B and 6C together show an orchestration sequence diagram illustrating the data flow of one embodiment of an API orchestrator system according to the current disclosure.

### DETAILED DESCRIPTION

Several aspects of systems, apparatus and methods for a unique communications system will now be presented with reference to the Figures, briefly described above, which may, for example, be implemented in an improved computing system or apparatus. These systems, apparatus and methods will be described in the following detailed description and illustrated in the accompanying drawings by various blocks, modules, components, circuits, steps, processes, algorithms, and/or the like (collectively referred to as “elements”). These elements may be implemented using hardware, software, or combinations thereof. Whether such elements are implemented as hardware or software depends upon the particular application and design constraints imposed on the overall system.

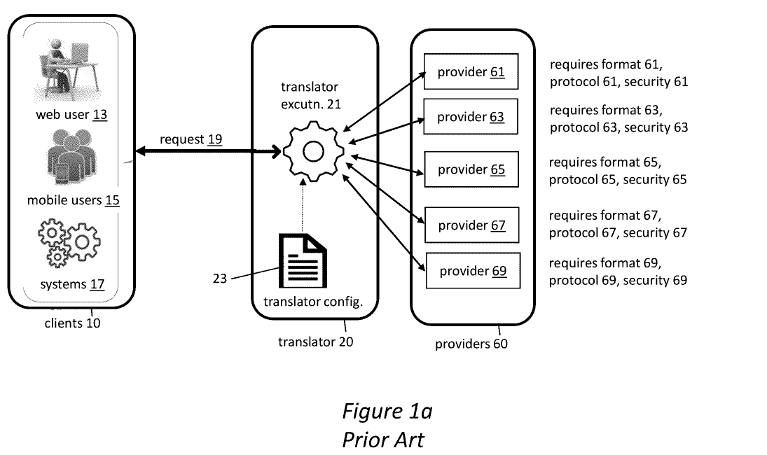

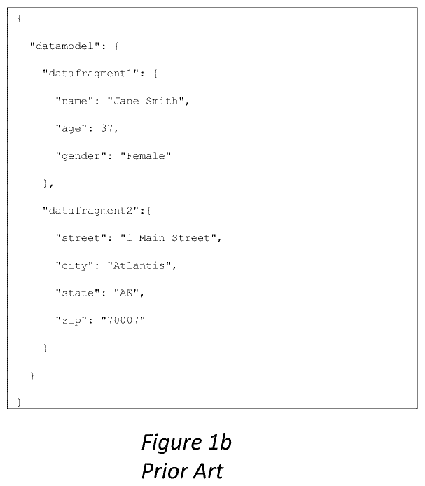

FIG. 1 is a functional block diagrammatic overview of a prior art translation system. As shown in FIG. 1, in prior art systems, a client 10 would like to interact with at least one of the providers 60; however, each client 10 and each provider 60 are most likely speaking a different electronic language. These languages differ in the types of data provided, the content of the data, the order of the data, and the “handshaking,” security, and protocol. A translator 20 would be required to create compatible communication between client 10 and provider 60.

There are different clients, such as web users 13, mobile users 15, and systems 17, which have different internal data flows and communication. Similarly, there are different providers 61, 63, 65, 67, and 69, which also communicate differently. Therefore, translator 20 will have to be able to convert the communication of each client 10 to be compatible with the communication of each provider 60 to which it is speaking. This also works in the opposite direction in which each message from a provider 60 must be converted into a format, protocol, and security compatible with client 10 to which the provider 60 is communicating/speaking. For example, provider 61 defines a format, protocol, and security with which it will connect/communicate (i.e., that it will “speak”) and understand. Anything else will cause an error or be ignored. Therefore, this results in a technical problem.

Similarly, providers 63, 65, 67, 69 each require respective formats, protocols and security information.

The basic translator typically employs translator execution device 21, configured by pre-stored translator configuration information 23. It routes the communication between a client 10 and the intended provider 60. It also converts the format, protocol, and security information to that of the intended provider 60. In this illustrative embodiment, this translator is “hard-wired,” i.e., it is only functional with specific architecture.

Having just three clients, 10 and five providers 60, will result in fifteen types of communication. As more clients, 10 and providers 60, are added, the complexity of the translator 20 increases geometrically. Each time another client 10 or provider 60 is added to the system, or a format, protocol, or security requirement is changed, it requires a modification to the translator configuration 23 and possibly the translator execution device 21. This situation can become very time-consuming, cumbersome, and costly to manage/maintain. Also, since a single translator 20 handles all of the translations, there can be no communications when it is being updated.

Communications between client 10 and provider 60 can sometimes get fairly involved. If the communication link were broken, typically, all of the information which has not yet been saved

```
{
  “datamodel”: {
    “datafragment1”: {
      “name”: “Jane Smith”,
      “age”: 37,
      “gender”: “Female”
    },
    “datafragment2”: {
      “street”: “1 Main Street”,
      “city”: “Atlantis”,
      “state”: “AK”,
      “zip”: “70007”
    }
  }
}
```

would be lost, causing the client 10 to start from the beginning again. This situation is very unpleasant to client 10 and results in lost data, and a waste of time and effort. Also, some of these configurations are relatively involved and difficult to set up again. If the connection is lost, these configurations may have to be rebuilt.

Prior art translators, such as an example illustrated above, were event or ‘operation-driven’ with pre-defined static integration models. These prior art translators or integration models resulted in specific operation(s) or event(s) in response to an input operation/event based on a fixed workflow. Inputs and outputs of operations/events of the translators were based on operation-specific, fixed subsets of a data model. The data interaction would be based on pre-defined data sets exchanged in data flow with a fixed sequence. Considering the example above, of a function that requires two data fragments—datafragment1 and datafragment2 which are subsets of a CDM as known in the prior art, the resources would be /translator/v1/ resource1 and /translator/v1/resource2 that accept a datafragment1 payload and followed by datafragment2 payload respectively. Corresponding provider APIs (not illustrated above) are invoked when the translator APIs are invoked.

In an embodiment according to this disclosure, with the orchestration system leveraging the top level resource entities in the CDM (for example “datamodel” in the example above), without adding resources with fixed data fragments or a predefined data flow sequence, the resources would be /orchestrator/v1/top-level-resource (datamodel) and it would accept datafragment1+datafragment2 or datafragment1 alone or datafragment2 alone. Providers would be invoked based on the data fragments provided to the /orchestrator/v1/top-level-resource (datamodel) API.

Since the prior art translators employed fixed subsets of the data model, they exhibited limited dynamic system configurability and data mapping.

Prior art translators did not provide complete decoupling of client interaction models from provider interaction models and did not support conflicting provider workflows. Using the same example above, if provider 1 accepts datafragment1 followed by datafragment2 then the resource model /translator/v1/resource1 and /translator/v1/resource2 would enable translator to invoke provider1's corresponding APIs. But, if provider2 accepts datafragment2 followed by datafragment1, the client workflow would be impossible to support by the translator as the data is arriving out of order. The current disclosure, leveraging the data aggregation strategy in the orchestrator and provider specific adapters with independent workflows can seamlessly support conflicting provider workflows.

Idempotency, i.e., producing the same result every time, was not guaranteed by the prior art translators. As the provider state is not maintained by the translator, the translator may invoke provider APIs on client request. This may result in an inconsistent state if the provider's APIs are not idempotent. The current system in this implementation according to the disclosure maintains provider state tied to a cryptographic hash of the request and hence will not make duplicate calls to the provider if a previous request is repeated. The cryptographic hash is used to compare contents of a request with adapters saved state to detect any change, in order to make a decision whether to repeat a workflow or not. If in a sequence of provider API calls a data element of a preceeding API is changed, then calls from that point are repeated to ensure the integrity of the collective state of the set of APIs is preserved.

The prior art translators also exhibited limited ability to auto-recovery from system failures. Due to these and other considerations, the system according to the disclosure was developed.

The system according to the disclosure leverages a canonical data model (CDM) and data aggregation-based integration strategy with runtime frameworks. A data-driven API orchestration engine (implemented in hardware, software or a combination thereof) aggregates input from client requests within a set/session and integrates it into a canonical model. The current system is dynamically configurable and employs data-driven workflow trigger points. Due to the architecture and system according to the disclosure there is a complete decoupling of data fragments, within the CDM, and data flow between the clients and the providers. Consider a domain with three complex elements in the data domain—A, B & C. For example, assume Provider1 accepts A in one API, followed by an API for B and a third one for C. Provider2 accepts B, followed by A and then C. A client who initially needs to integrate only with Provider 1 would find is easiest to follow the A->B->C data flow. If there is tight coupling with the client and provider, the client would not be able to integrate with Provider2 which has the B->A->C data flow. The proposed system solves this problem by the orchestration engine (orchestration engine and “orchestrator” and “orchestration module” are used herein interchangeably), inspecting the data to decide which provider API to call. When the client which has implemented the A->B->C invokes the orchestrator, the orchestrator will invoke only Provider1 when only A is provided. When B arrives, the orchestrator will invoke Provider1 with B, and then invoke Provider2 with B followed by A. There is also a complete decoupling of client state management and data representation from provider state management.

The current system according to the disclosure implements dynamic, templatized data mappers from the canonical data model to provider data models, including provider exception and error responses to data defined in the canonical data model. The disclosed current system provides automatic recovery from intermediate system errors from providers with idempotent provider state management, including partial success situations. Since the system according to the disclosure has a modular design, providers can be dynamically added or removed from the runtime environment.

The disclosed system is designed to configure client-specific, provider-agnostic workflow steps in the orchestration system and is designed to consume messages from and publish events to clients, providers, and intermediaries. The design separates the client requirements and configurations in the orchestration system and the provider requirements and configurations in the adapters. The client configurations including routing and pre processing, post processing, and persistence functions are chosen based on authenticated client's system identifier. Examples of pre-processing functions are client/customer eligibility checks, logging, audting, and data feeds. An example of post-processing functions are data feeds, auditing and notifications.

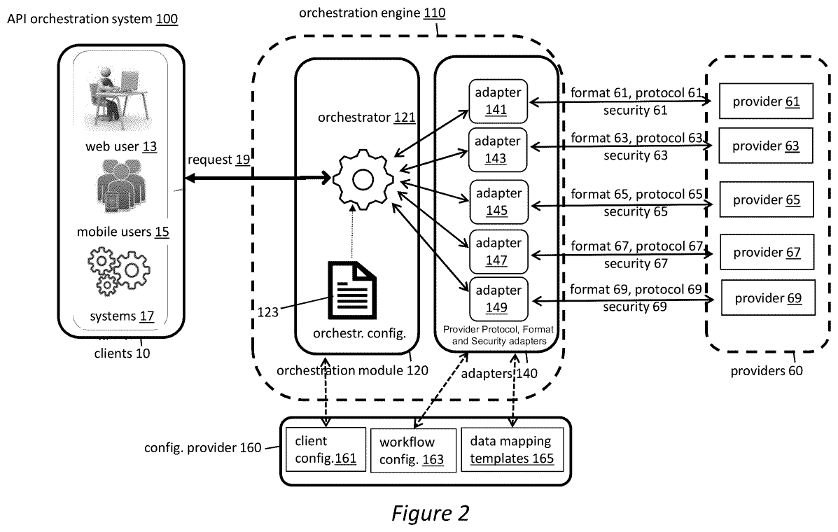

FIG. 2 is a functional block diagrammatic overview of an API orchestration system according to one aspect of the present disclosure. The functions of translators, implemented in hardware, software, or a combination thereof, can be separated into those which are common and those which are specific to each individual provider. The current system is designed to incorporate the common orchestration functions into an orchestration engine 110 that comprises orchestration module 120, and the provider-specific functionality into a plurality of adapters 141, 143, 145, 147, 149 (collectively “140”). Each adapter is designed to communicate with a specific provider 61, 63, 65, 67, 69.

The orchestration module 120 (it should be appreciated that all of the components/modules described herein may be implemented in hardware, software, or a combination thereof), is responsible for client configuration and workflows, creating and managing an aggregated data model, client specific state management and integrating the adapters 140.

Orchestration module 120 has the function of specifying and implementing the ‘language’ (data formats, data schema, protocol, security) that will be used by the clients 10 and implementing a universal model (a canonical data model (CDM)) encompassing all data fragments of the clients 10. It also has the function of analyzing the input from a client 10 and information from the provider to determine which provider 60 should be linked to the client 10. It then links the client 10 to the selected provider 60. Communications between the clients 10 and the providers 60 are generally two-way communication, but they can be one-way communication in alternative embodiments. Typically these communications involve many messages.

In FIG. 2, clients 10, include, for example, web user 13, who tries to communicate with a service provider, such as service provider 67. The orchestration module 120 reviews client functionality needs during system setup and configures provider routes and other cross cutting functions such as data feeds as client config 121 that will be a part of orchestrator config (configuration information) 123. The orchestration module 120 then sets up a standard for communication with the clients with authorization setup with clients and any new providers. The authorization between client and orchestrator would be similar to login security, should be dependent on the API provider capabilities and would be encapsulated in a logical or physical layer in between client and orchestrator. One example embodiment in an architecture with an API gateway could be supported by most API gateway implementations.

An orchestrator 121 is implemented to operate according to orchestrator configuration information 123.

Orchestration module 120 uses client configuration information from client configuration device 161 of a configuration provider 160. The orchestration module 120 receives information to identify which providers are eligible to communicate with web user 13. In this illustrative embodiment it is determined that provider 67 is eligible.

The corresponding adapter 147 receives workflow and data format requirements defined by the provider 67. These are provided and stored in a corresponding workflow configuration device 163 and data mapping template device 165, and are used to convert data format, protocol, and security of the orchestration module 120 to that of provider 67.

Orchestration module 120 communicates through adapter 147, specifically configured to communicate with a corresponding format, protocol, and security information, with the provider 67.

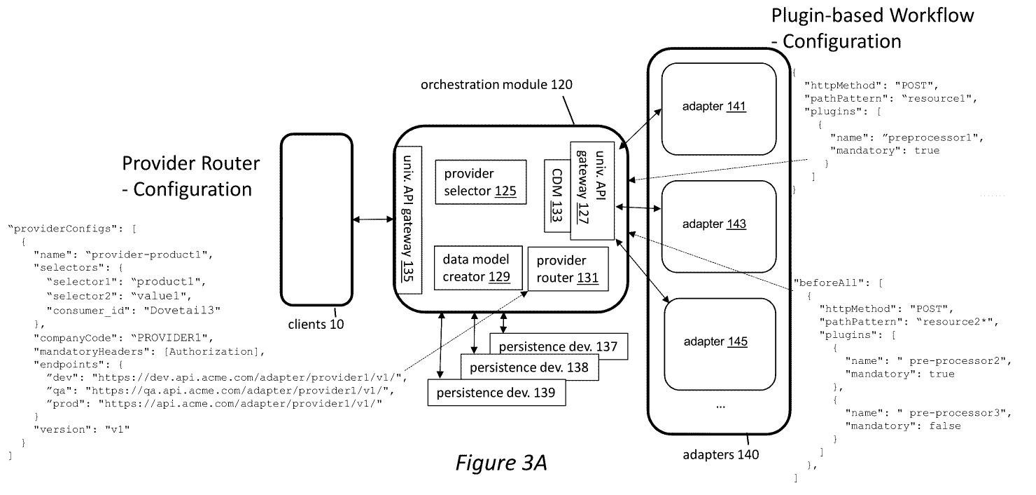

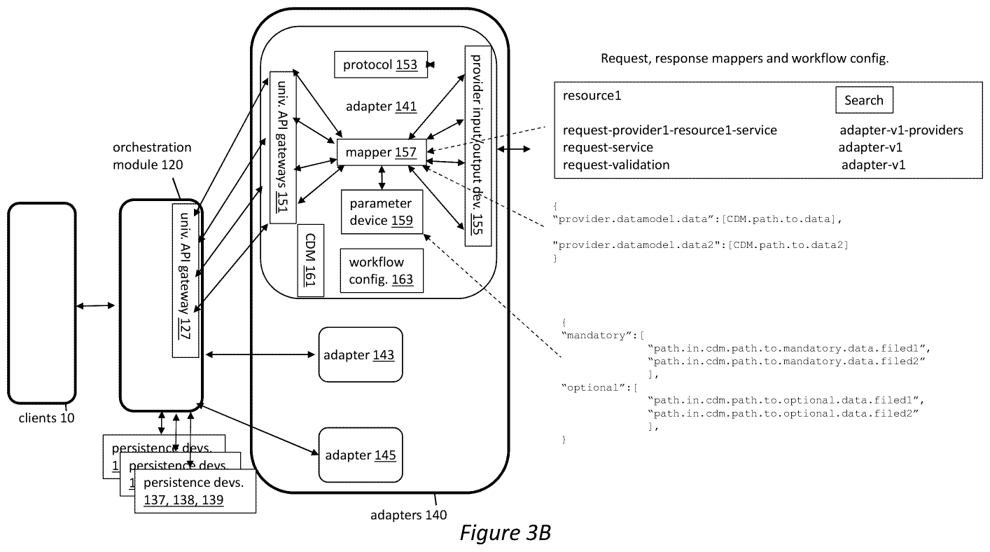

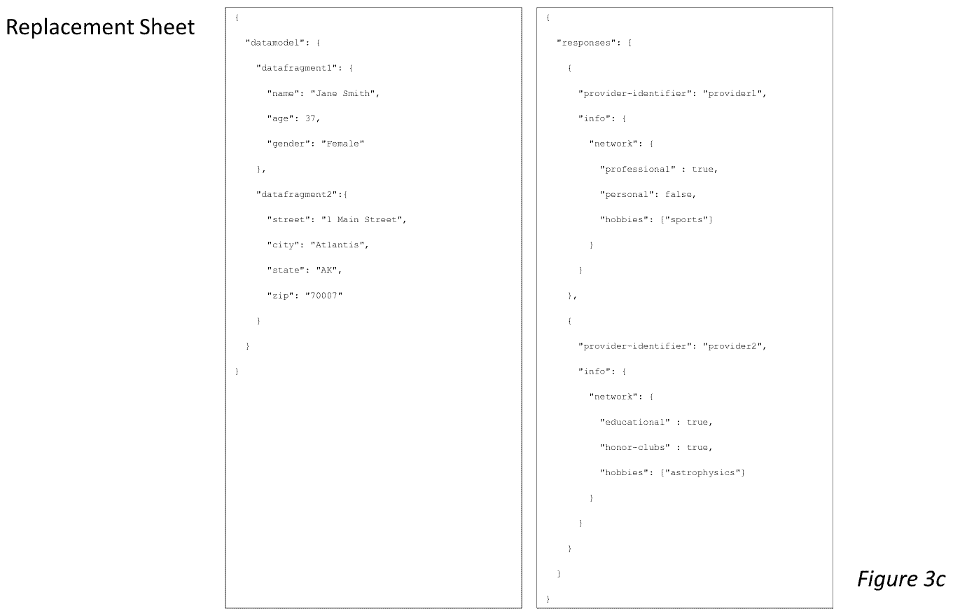

FIG. 3A is a partial functional block diagram of the API orchestration system according to the disclosure showing greater detail and operation of the orchestration module 120. A provider selector 125 determines which provider 60 is eligible to communicate with the web user 13 from the carrier selector configuration provided.

A data model creator 129 creates the CDM 133, which aggregates the data fragments from clients 10. The data model creator 129 repeatedly aggregates the data formats of the clients 10. This repeated aggregation allows the cumulative data driven workflows to the providers from the adapters and the flexibility to encapsulate conflicting workflows between providers within provider adapters. The CDM defines a common way of communicating with the clients 10 and with adapters 140.

Orchestration module 120 further includes a universal API gateway 127 facing the adapters 140. It also includes a plurality of input APIs with standardized data formats, protocol, and security. A plurality of universal API gateways implemented in the orchestration module 120 incorporate standardized input/output data formats, protocol, and security. Therefore, any of the adapters 140 employing the same standardized input/output format, protocol, and security can plug into the orchestration module 120. These standardized API gateways allow the orchestration system 100 to be modular with plug-in adapters 140. This modular design allows the system to accept new providers by creating an adapter specific to each new provider and plugging them into the orchestration module 120 of the system.

The functioning of the provider selector 125 may be changed by dynamically changing its configuration. An intelligent router 131 acquires information about the clients 10 and the providers 60 and determines which providers 60 are eligible to be connected to a given client. Similarly, the functioning of the intelligent router 131 may be changed by changing its configuration. The plug-in-based workflow configurations of the orchestration module 120 may be changed to change its functionality.

FIG. 3B is a partial functional block diagram of the API orchestration system showing a more detailed view of the adapters 140. Adapters 140 are responsible for provider 60 workflows, orchestrations, and data mapping from/to the canonical data model (CDM), including specification enforcement.

The adapters 140 have universal API gateways 151 compatible with the universal API gateways 127 of the orchestration module 120. As described above, this allows additional adapters 140 to easily be added to the system and interface with the orchestration module 120.

The adapters 140 employ a mapping device 157, which maps data and data fields received at its input (as defined in the CDM) to the data structure of a provider at its output.

The adapter 140 includes a protocol device 153 that controls data transmission according to a protocol compatible with the provider's protocol through a provider's input/output device 155 when communicating to a provider. The protocol device 153 controls data transmission through the universal API gateway 151 according to a protocol compatible with the protocol of the orchestration module 120 when transmitting data in the opposite direction.

The adapter 140 also includes a parameter device 159, which is tasked with inserting required parameters into the communications messages. Upon the command of the clients or provider, optional parameters may also be included in the communication messages.

Interfaces from an adapter 140 to its corresponding provider 60 are based on provider specifications.

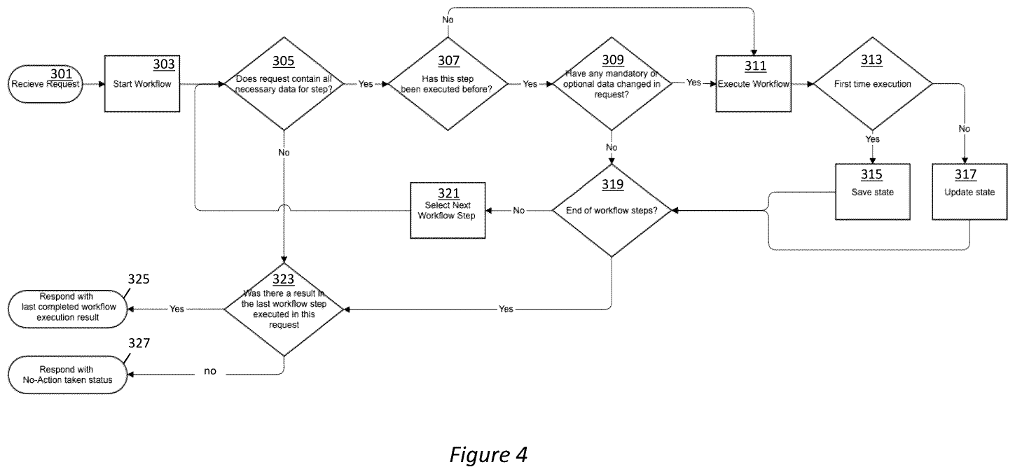

The adapter 140 also includes the workflow configuration device 163 that deteremines data model driven trigger points and preconditions for workflow step execution as shown in FIG. 4.

Persistence devices 137, 138 139 are used to continually save and stores the state of the orchestrator module, one device per client. The persistent state includes the workflow execution steps representing aggregated results across all providers. Persistence devices 171 store the state of adapters 140, one each per adapter. The state includes workflow execution results from all providers 60, request management for idempotency needs, and canonical data model representation of provider API payloads.

The example below shows sample requests to an orchestrator, according to the disclosure, to find social networks for a person. The request has the person's demographic information and each provider responds with the types of networks that the person has on the provider's platform.

As illustrated below, the request (box/flow on the left-hand side), represents a request for demographic information, which includes name, age and gender, in datafragment1. Datafragment2 includes street, city and state addresss, as well as zip code information.

In the example below, each provider responds (box/flow on the right-hand side), with the types of networks that the person has on the provider's platform. For example, provider 1 returns professional and personal networks, and provider 2 returns educational and honorary membership networks.

```
{
  “datamodel”: {
    “datafragment1”: {
      “name”: “Jane Smith”,
      “age”: 37,
      “gender”: “Female”
    },
    “datafragment2”: {
      “street”: “1 Main Street”,
      “city”: “Atlantis”,
      “state”: “AK”,
      “zip”: “70007”
    }
  }
}
```

```
{
  “responses”: [
    {
      “provider-identifier”: “provider1”,
      “info”: {
        “network”: {
          “professional” : true,
          “personal”: false,
          “hobbies”: [“sports”]
        }
      }
    },
    {
      “provider-identifier”: “provider2”,
      “info”: {
        “network”: {
          “educational” : true,
          “honor-clubs” : true,
          “hobbies”: [“astrophysics”]
        }
      }
    }
  ]
}
```

FIG. 4 is a flowchart illustrating execution of workflow steps of one embodiment of an API orchestration system according to the current disclosure. The following actions are executed for each step defined in provider workflow:

A request is received at step 301.

At step 303, the system begins executing workflow commands.

A determination is made if mandatory data elements are present in request payload.in step 305.

If all of the mandatory data elements are not present, (“no”), then execution of the workflow stops, and processing continues at block 323.

If all of the mandatory data elements are present, (“yes”), then, in block 307, it is determined if this workflow step has been previously executed.

If the workflow step has not been previously executed, (“no”), then the workflow step is exected in block 311.

If the workflow step has been previously executed, (“yes”), then a decision is made to determine if any mandatory or optional data has changed in the request.

If any mandatory or optional data has changed in the request, (“yes”), then block 311 is performed which executes the workflow step.

If no mandatory or optional data has changed in the request, (“no”), then, in step 319, it is determined if all of the workflow steps have been completed.

If all of the workflow steps have not yet been completed (“no”), then the next workflow step is selected in block 321 and processing continues at block 305 with the next workflow step.

If all of the workflow steps have been completed (“yes”), then it is determined if there was a result to the last workflow step executed in the current request.

If there is a result to the last workflow step executed in the current request (“yes”), then a response is created and sent with the last workflow step execution result in block 325.

If there was not a result to the last workflow step executed in the current request (“no”), then a response is created and sent indicating a ‘No Action Taken’ status in block 327.

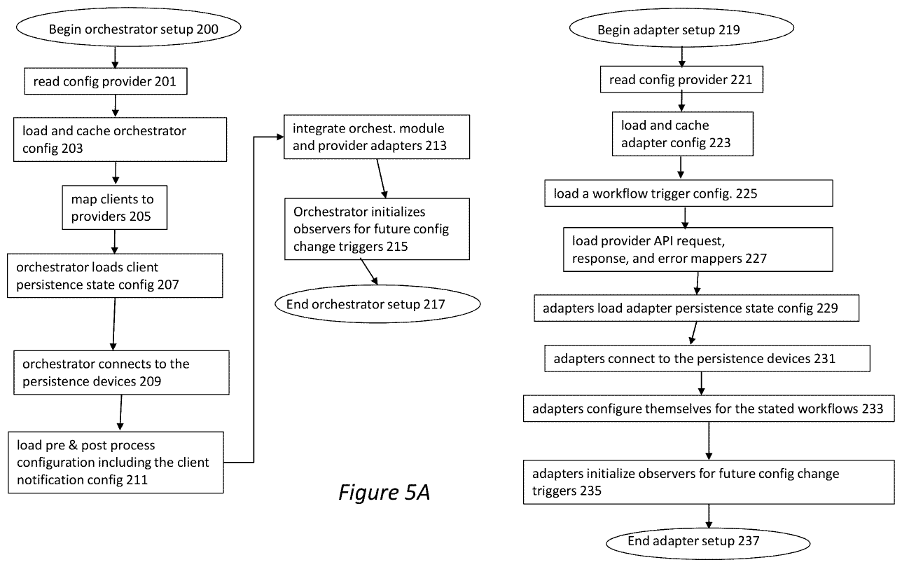

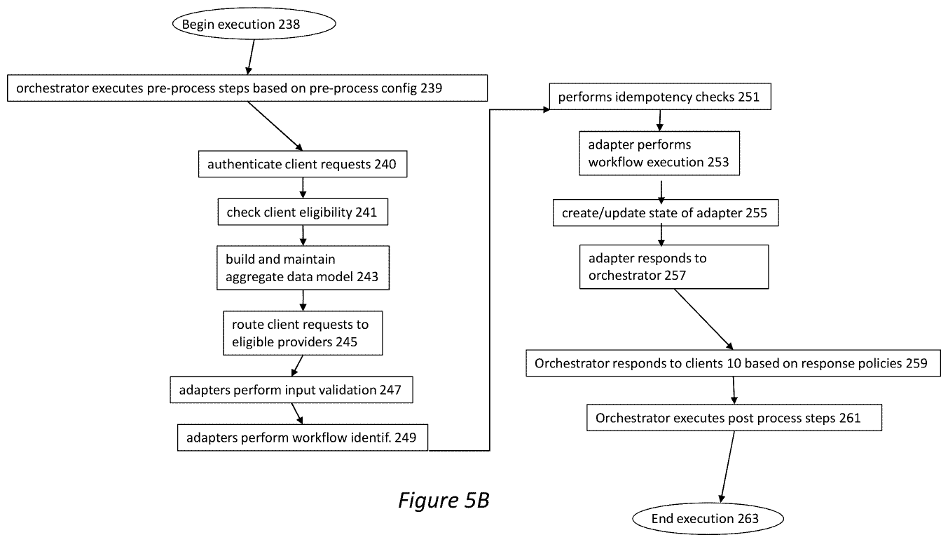

FIG. 5A is a flowchart illustrating the system initialization and scheduled periodic, configuration reload functions of one embodiment of an API orchestration system according to the current disclosure. These functions will be described further in connection with FIGS. 2-5A.

System Design—The orchestration module 120 system design is determined.

In step 200, the setup of the orchestrator module 120 is initiated.

In step 201, the orchestrator module 120 reads the config provider 160 on startup as well as at periodic intervals subsequently.

In step 203, the orchestration module 120 loads and caches the orchestration configurations related to clients, providers, persistence locations and common functions from the configuration provider 160.

System Set-Up—Orchestrator Module—in step 205, the orchestration module 120 interprets the configuration information indicating supported providers 60 and maps clients 10 to the supported providers 60.

In step 207, the orchestration module 120 loads the client persistence state management configuration.

In step 209, the orchestration module 120 connects to the persistence devices.

In step 211, the orchestration module 120 loads the pre and post process configuration including the client notification configuration.

In step 213, the orchestration module 120 integrates with provider-adapters 140.

In step 215, the orchestration module 120 initializes observers for future config change triggers.

The orchestration module 120 setup ends in step 217.

System Design—The provider adapters 140 system design is determined.

In step 219, the setup of the provider adapters 140 is initiated.

In step 221, the provider adapters 140 read the config provider 160 on startup as well as at periodic intervals subsequently.

In step 223, the provider adapters 140 load and cache the configurations related to workflow compositions with provider APIs, workflow trigger points, and API request, response, and error mapping and persistence from the configuration provider 160.

System Set-Up—Provider Adapters

In step 225, provider adapters 140 load a workflow trigger configuration. A workflow trigger configuration defines the data set required to start a workflow step. The configuration would be composed of data elements that are mandatory, and an optional set that if changed should result in re-execution of a workflow.

In step 227, provider adapters 140 load the provider API request, response, and error mappers.

In step 229, the provider adapters 140 load the adapter persistence state management configuration, which includes the provider state and adapter responses.

In step 231, the provider adapters 140 connect to the persistence devices.

In step 233, the provider adapters 140 configure themselves for the stated workflows to be executed.

In step 235, the provider adapters 140 initialize observers for future config change triggers.

System Initialization

The provider adapters 140 setup then ends in step 237.

FIG. 5B is a flowchart illustrating the request/response functions of one embodiment of an API orchestrator system according to the current disclosure.

System Execution

The process starts at step 238.

In step 239, orchestration system components execute the pre-process steps based on the pre-process configuration from the configuration provider 160.

In step 240, the client requests are authenticated and authorized by the orchestration module 120.

In step 241, client eligibility checks are then performed by orchestration module 120.

In step 243, the data model creator 129 of the orchestration module 120 builds and maintains an aggregate data model.

In step 245, the intelligent router 131 of the orchestration module 120 routes requests to eligible provider adapters 140 filtered by configured selectors.

In step 247, each provider adapter 140 performs input validation based on provider needs in step 233.

In step 249, each adapter 140 then performs workflow identification.

In step 251, each adapter 140 also performs idempotency checks.

In step 253, each adapter 140 performs workflow execution. The workflow execution may include:
reading current provider state for request/session/persistent scope,
creating provider requests, data mapping & execution,
handling provider responses, including data mapping to CDM or error response mapping,
updating the local representation of the provider state,
managing request state to enable scheduled or next-request retry, if there was a communication error, or
performing workflow orchestration.

In step 255, The adapters 140 create/update the adapter state.

In step 257, adapter 140 returns a response in canonical data format to the orchestration module 120.

In step 259, the orchestrator 120 aggregates successes and errors and responds to clients 10 based on response policies. These response policies may be to:
send a response message if there is at least one success, or
send a different response message if there are errors from all providers.

In step 261, the orchestrator 120 executes the post process steps which includes notifications.

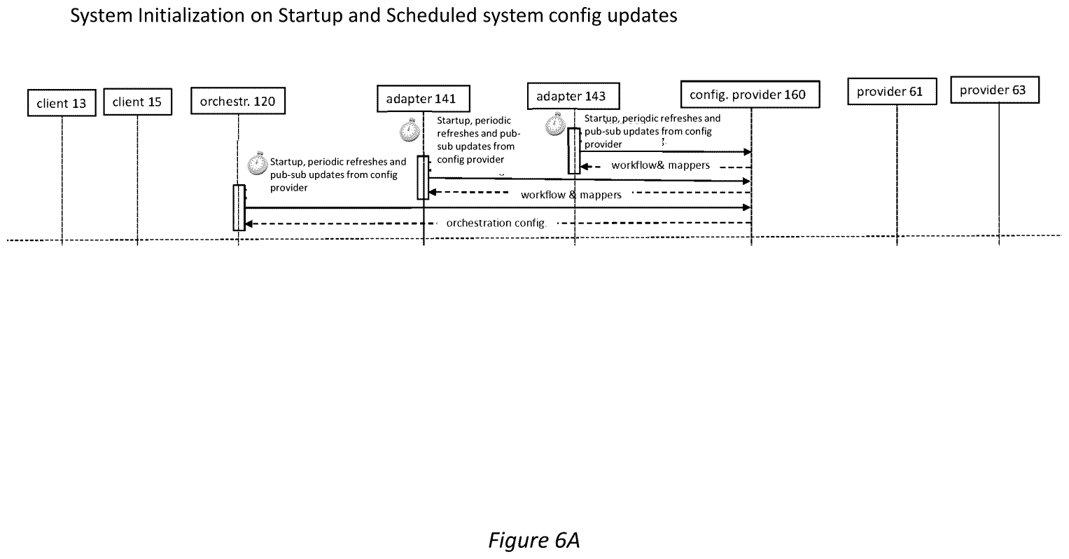

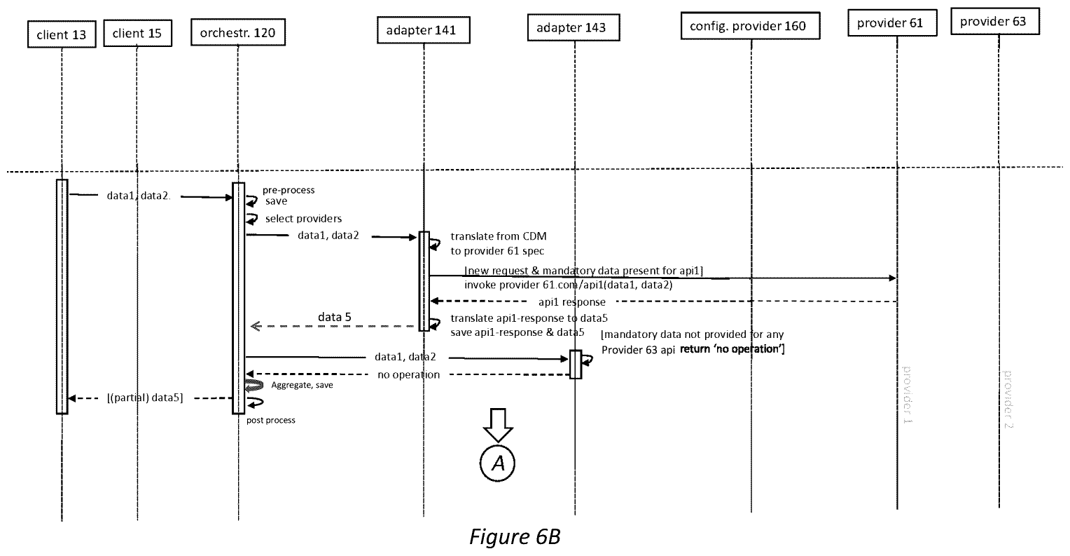

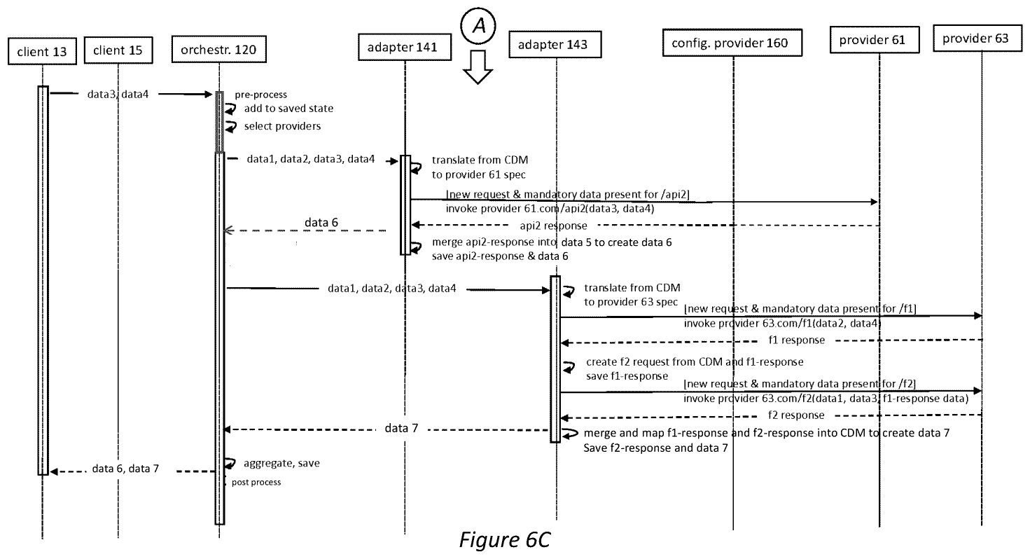

FIGS. 6A, 6B and 6C together show orchestration sequence diagrams illustrating the data flow of one embodiment of an API orchestration system according to the disclosure. These sequences or timing diagrams generally follow the flowcharts of FIGS. 5A and 5B, but illustrate relative timing of the sequences discussed.

Clients 13 and 15 were chosen for this example, but it should be appreciated that such sequences and timing may be in regard to any of the clients 10 described above.

Providers 61 and 63 were chosen for this example; however, it should be appreciated that this process applies to any of the providers 61-69 shown in FIGS. 1 and 2, and described herein.

As illustrated in FIGS. 6A, adapter 143 reads the configuration provider 160 for configuration related to workflow and mappers. Reading from the configuration provider 160 occurs during startup as well as during periodic refreshes and pub-sub updates (i.e., “publisher/subscriber updates”) received from configuration provider 160. The “pub-sub” pattern, also known as Publish/Subscribe pattern, is a system architecture design pattern that provides a framework for exchanging messages between publishers of messages and subscribers (intended to receive messages). This pattern involves the publisher and the subscriber relying on a message broker that relays messages from the publisher to the subscribers. In this embodiment of the disclosure, it means that when there is a change made in the “configuration provider 160”, it publishes the config updates and the orchestrator 120 and adapters 140 receive those updates as subscribers.

Adapter 143 then loads and caches the configurations related to workflow compositions with provider APIs, workflow trigger points, and API request, response, and error mapping and persistence from the configuration provider 160.

Adapter 141 reads the configuration provider 160 for configuration related to workflow and mappers. Reading from the configuration provider 160 occurs during startup as well as during periodic refreshes and pu-sub updates received from configuration provider 160.

Adapter 141 then loads and caches the configurations related to workflow compositions with provider APIs, workflow trigger points, and API request, response, and error mapping and persistence from the configuration provider 160.

Orchestrator 120 reads the orchestration configuration from the configuration provider 160. Reading from the configuration provider 160 occurs during startup as well as during periodic refreshes and pub-sub updates received from configuration provider 160.

Orchestrator 120 then loads and caches the orchestration configurations related to clients, providers, persistence locations and common functions from the configuration provider 160. This ends the setup phase. The system is now ready to operate.

In FIG. 6B, a client 13 sends a request having data1 and data2 to orchestrator 120.

Orchestrator 120 then executes pre-process steps based on pre-process configuration, saves data1, data2 and selects providers.

Data1, data2 is then sent from orchestrator 120 to adapter 141.

Adapter 141 then translates from the canonical data model (CDM) to data format, protocol and security information required by provider 61.

Adapter 141 creates and sends a new request with mandatory data present for api1 to provider 61 in the data format, protocol and security information required by provider 61 to invoke provider 61. com/api1(data1, data2).

Provider 61 then returns the api1 response to adapter 141.

Adapter 141 then translates ‘api1-response’ from provider 61 into ‘data5’ and saves them.

Adapter 141 then returns “data5” to the orchestrator 120

The initial request with ‘data1, data2’ is also sent to other adapters, such as adapter 143. If an adapter, such as adapter 143, is associated with a provider which cannot process the ‘data1, data2’ request, it returns a notification of ‘no operation’. Basically, if the mandatory data is not provided for a provider 63 api, the provider 63 api returns a message of ‘no operation’ to orchestrator 120.

Orchestrator 120 then aggregates and saves the reposnses from adapter 141 and 143.

Orchestrator 120 then executes the post-process steps based on the post-process configuration.

Orchestrator 120 then sends partial data5 back to client 13.

In FIG. 6C, client 13 sends a data request ‘data3, data4’ to orchestrator 120.

Orchestrator 120 then executes pre-process steps based on pre-process configuration, saves/adds the request(“data3, data4”) to the previously saved state and selects eligible providers.

A message with ‘data1, data2, data3, data4’ is then sent from orchestrator 120 to adapter 141.

Adapter 141 translates the message with ‘data1, data2, data3, data4’ using the CDM into the communication specifications of provider 61.

A new request with the mandatory data present for a function/api2 is created and sent from adapter 141 to provider 61.

This invokes provider 61 to invoke 61.com/api2(data3, data4).

Provider 61 then returns api2 response' to adapter 141.

Adapter 141 then merges api2-response' with “data5” to create “data6”, and saves them.

Adapter 141 then returns “data6” to the orchestrator 120.

The message with ‘data1, data2, data3, data4’ is also sent from orchestrator 120 to adapter 143.

Adapter 143 translates the message with ‘data1, data2, data3, data4’ using the CDM into the communication specifications of provider 63.

A new request with the mandatory data present for a function/f1 is created and sent from adapter 143 to provider 63.

This request invokes ‘provider 63.com/f1(data2, data4).

Provider 63 then returns ‘f1 response’, which is the output of function ‘f1’, to adapter 143.

Adapter 143 then creates an ‘f2 request’ from CDM and the ‘f1 response’, and saves the ‘f1 response’.

A new request with the mandatory data present for a function ‘/f2’ is created and sent from adapter 143 to provider 63.

This request invokes ‘provider 63.com/f2(data1, data3, f1 response data).

Provider 63 then returns ‘f2 response’, which is the output of function ‘f2’, to adapter 143.

Adapter 143 then merges and maps the ‘f1 response’ and “f2 response” into CDM to create “data7”, and saves them.

Adapter 143 then sends ‘data7’ to orchestrator 120.

Orchestrator 120 then aggregates data and saves the responses from adapter 141 and 143.

Orchestrator 120 then executes the post-process steps based on the post-process configuration.

Orchestrator 120 then sends a message ‘data6, data7’ back to client 13 as the system response.

The current API orchestration system has the advantage of having complete decoupling of client and provider integration models. It also takes advantage of a data-aggregation strategy to maintain the canonical data model. The current API orchestration system operates on dynamic subsets of the canonical data model. It can also dynamically change provider integration workflows with data-driven triggers instead of event/operation level triggers.

The current API orchestration system has inherent idempotency, independent of provider capabilities. he system in this implementation maintains provider state tied to a cryptographic hash of the request and hence will not make duplicate calls to the provider. If in a sequence of provider API calls a data element of a preceeding API is changed, then calls from that point are repeated to ensure the integrity of the collective state of the set of APIs is preserved.

The current system includes recovery from transient provider failures that are automatically performed without the need for any input from the clients.

Various aspects of the disclosure have been described fully above with reference to the accompanying drawings. This disclosure may, however, be embodied in many different forms and should not be construed as limited to any specific structure or function presented throughout this disclosure. Rather, these aspects are provided so that this disclosure will be thorough and complete, and will fully convey the scope of the disclosure to those skilled in the art. Based on the teachings, one skilled in the art should appreciate that the scope of the disclosure is intended to cover any aspect of the disclosure disclosed, whether implemented independently of or combined with any other aspect of the disclosure. For example, an apparatus or system may be implemented or a method may be practiced using any number of the aspects set forth. In addition, the scope of the disclosure is intended to cover such an apparatus, system or method which is practiced using other structure, functionality, or structure and functionality in addition to or other than the various aspects of the disclosure set forth. It should be understood that any aspect of the disclosure disclosed may be embodied by one or more elements of a claim.

The word “exemplary” or “illustrative” are used herein to mean “serving as an example, instance, or illustration.” Any aspect described herein as “exemplary” or “illustrative” is not necessarily to be construed as preferred or advantageous over other aspects.

Although particular aspects are described herein, many variations and permutations of these aspects fall within the scope of the present disclosure. Although some benefits and advantages of the preferred aspects are mentioned, the scope of the present disclosure is not intended to be limited to particular benefits, uses or objectives. Rather, aspects of the present disclosure are intended to be broadly applicable to different technologies, system configurations, networks and protocols, some of which are illustrated by way of example in the figures and in the following description of the preferred aspects. The detailed description and drawings are merely illustrative of the present disclosure rather than limiting, the scope of the present disclosure being defined by the appended claims and equivalents thereof.

As used herein, the term “determining” encompasses a wide variety of actions. For example, “determining” may include calculating, computing, processing, deriving, investigating, looking up (e.g., looking up in a table, a database or another data structure), ascertaining and the like. Additionally, “determining” may include receiving (e.g., receiving information), accessing (e.g., accessing data in a memory) and the like. Furthermore, “determining” may include resolving, selecting, choosing, establishing, and the like.

As used herein, a phrase referring to “at least one of” a list of items refers to any combination of those items, including single members. As an example, “at least one of: a, b, or c” is intended to cover: a, b, c, a-b, a-c, b-c, and a-b-c.

The various illustrative logical blocks, modules and circuits described in connection with the present disclosure may be implemented or performed with a processor specially configured to perform the functions discussed in the present disclosure. The processor may be a neural network processor, a digital signal processor (DSP), an application specific integrated circuit (ASIC), a field programmable gate array signal (FPGA) or other programmable logic device (PLD), discrete gate or transistor logic, discrete hardware components or any combination thereof designed to perform the functions described herein. Alternatively, the processing system may comprise one or more neuromorphic processors for implementing the neuron models and models of neural systems described herein. The processor may be a microprocessor, controller, microcontroller, or state machine specially configured as described herein. A processor may also be implemented as a combination of computing devices, e.g., a combination of a DSP and a microprocessor, a plurality of microprocessors, one or more microprocessors in conjunction with a DSP core, or such other special configuration, as described herein.

The steps of a method or algorithm described in connection with the present disclosure may be embodied directly in hardware, in a software module executed by a processor, or in a combination of the two. A software module may reside in storage or machine readable medium, including random access memory (RAM), read only memory (ROM), flash memory, erasable programmable read-only memory (EPROM), electrically erasable programmable read-only memory (EEPROM), registers, a hard disk, a removable disk, a CD-ROM or other optical disk storage, magnetic disk storage or other magnetic storage devices, or any other medium that can be used to carry or store desired program code in the form of instructions or data structures and that can be accessed by a computer. A software module may comprise a single instruction, or many instructions, and may be distributed over several different code segments, among different programs, and across multiple storage media. A storage medium may be coupled to a processor such that the processor can read information from, and write information to, the storage medium. In the alternative, the storage medium may be integral to the processor.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

The functions described may be implemented in hardware, software, firmware, or any combination thereof. If implemented in hardware, an example hardware configuration may comprise a processing system in a device. The processing system may be implemented with a bus architecture. The bus may include any number of interconnecting buses and bridges depending on the specific application of the processing system and the overall design constraints. The bus may link together various circuits including a processor, machine-readable media, and a bus interface. The bus interface may be used to connect a network adapter, among other things, to the processing system via the bus. The network adapter may be used to implement signal processing functions. For certain aspects, a user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further.

The processor may be responsible for managing the bus and processing, including the execution of software stored on the machine-readable media. Software shall be construed to mean instructions, data, or any combination thereof, whether referred to as software, firmware, middleware, microcode, hardware description language, or otherwise.

In a hardware implementation, the machine-readable media may be part of the processing system separate from the processor. However, as those skilled in the art will readily appreciate, the machine-readable media, or any portion thereof, may be external to the processing system. By way of example, the machine-readable media may include a transmission line, a carrier wave modulated by data, and/or a computer product separate from the device, all which may be accessed by the processor through the bus interface. Alternatively, or in addition, the machine-readable media, or any portion thereof, may be integrated into the processor, such as the case may be with cache and/or specialized register files. Although the various components discussed may be described as having a specific location, such as a local component, they may also be configured in various ways, such as certain components being configured as part of a distributed computing system.

The machine-readable media may comprise a number of software modules. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a special purpose register file for execution by the processor. When referring to the functionality of a software module below, it will be understood that such functionality is implemented by the processor when executing instructions from that software module. Furthermore, it should be appreciated that aspects of the present disclosure result in improvements to the functioning of the processor, computer, machine, or other system implementing such aspects.

If implemented in software, the functions may be stored or transmitted over as one or more instructions or code on a computer-readable medium. Computer-readable media include both computer storage media and communication media including any storage medium that facilitates transfer of a computer program from one place to another.

Further, it should be appreciated that modules and/or other appropriate means for performing the methods and techniques described herein can be downloaded and/or otherwise obtained by a user terminal and/or base station as applicable. For example, such a device can be coupled to a server to facilitate the transfer of means for performing the methods described herein. Alternatively, various methods described herein can be provided via storage means, such that a user terminal and/or base station can obtain the various methods upon coupling or providing the storage means to the device. Moreover, any other suitable technique for providing the methods and techniques described herein to a device can be utilized.

It is to be understood that the claims are not limited to the precise configuration and components illustrated above. Various modifications, changes, and variations may be made in the arrangement, operation, and details of the methods and apparatus described above without departing from the scope of the claims.

1. An application program interface (API) orchestration system that facilitates communication between at least one of a plurality of client systems with at least one of a plurality of provider systems, comprising:
an orchestration module coupled to a client system, comprising:
a provider selector that receives input from the client system and associates a set of provider systems (1 to n) with the client system based upon the input received from the client system, and links the client system to a selected provider system,
an intelligent router adapted to determine eligible provider systems for each client system and to route communications between the client system to a selected eligible provider system,
a data model creator which aggregates each input from the client system and creates a canonical data model (CDM) encompassing data fragments from the client system, and
a plurality of orchestration module universal input and output interfaces, each adapted to communicate information in a predetermined standardized data format and protocol;
a plurality of adapters receiving a set of security, data format, and protocol requirements defined by an associated provider system and communicating information with the associated provider system in a manner that is compatible with the received set of security, data format, and protocol requirements, each adapter comprising:
a plurality of adapter universal input and output interfaces that are compatible with and connected to the orchestration module universal input and output interfaces of the orchestration module;
a parameter device selecting workflow steps to be executed based on data in a request adhering to the CDM;
a mapper that maps data fields of the CDM and added parameters with corresponding data fields of the provider system resulting in the data format of the provider system,
a protocol device that receives and stores protocol requirements from the provider system, and
a provider input/output device coupled to the mapper that receives provider system data in the data format of the provider system and communicates the provider system data according to the protocol requirements stored in the protocol device.

2. The API orchestration system of claim 1, wherein the parameter device runs pre-stored workflow steps according to a pre-stored workflow configuration that adds mandatory and optional parameters to communications between the client system and the provider system.

3. The API orchestration system of claim 1, further comprising a plurality of persistence devices, one per adapter, having a power source independent from the API orchestration system.

4. The API orchestration system of claim 1, further comprising a plurality of persistence devices that save a current state of provider interactions and adapter state returned to the orchestration system.

5. The API orchestration system of claim 1, further comprising a plurality of persistence devices that save a current state of provider interactions and adapter state returned to the orchestration system, and load the adapter state into the adapters on a restart of the adapters.

6. The API orchestration system of claim 1, further comprising a plurality of persistence devices, each coupled to a single client system, each of the plurality of persistence devices having a power source that is independent of that of the API orchestration system, the power source being coupled to the orchestration module and interactively storing a current state of the orchestration module, and loading the stored current state of the orchestration module into the orchestration module when there has been a restart of the orchestration module.

7. The API orchestration system of claim 1, further comprising additional universal API gateway interfaces in the orchestration module to allow adapters each specific to an additional provider system to be added that communicate through the universal API Gateway.

8. The API orchestration system of claim 1, further comprising a configuration provider that receives and stores configuration information from the orchestration module and adapters and provides the configuration information back to the orchestration module and the adapters upon start-up and upon any updates of orchestration system configuration.

9. An application program interface (API) orchestration system that facilitates communication between at least one of a plurality client systems with at least one of a plurality of provider systems, comprising:
an orchestration module coupled to a client system, comprising:
a provider selector that receives input from the client system and associates a set of provider systems (1 to n) with the client system based upon the input received from the client system, and links the client system to a selected provider system,
an intelligent router adapted to determine eligible provider systems for each client system and to route communications between the client system to a selected eligible provider system,
a data model creator which aggregates each input from the client system and creates a canonical data model (CDM) encompassing data fragments from the client system, and
a plurality of orchestration module universal input and output interfaces, each adapted to communicate information in a predetermined standardized data format and protocol;
a plurality of adapters receiving a set of security, data format, and protocol requirements defined by an associated provider system and communicating information with the associated provider system in a manner that is compatible with the received set of security, data format, and protocol requirements; and
wherein the plurality of adapters includes a mapper that maps data fields of the CDM and added parameters with corresponding data fields of the provider system resulting in the data format of the provider system.

10. The application program interface (API) orchestration system of claim 9, wherein each adapter comprises:
a plurality of adapter universal input and output interfaces that are compatible with and connected to the orchestration module universal input and output interfaces of the orchestration module;
a parameter device selecting workflow steps to be executed based on data in a request adhering to the CDM;
a protocol device that receives and stores protocol requirements from the provider system, and
a provider input/output device coupled to the mapper that receives provider system data in the data format of the provider system and communicates the provider system data according to the protocol requirements stored in the protocol device.

11. The application program interface (API) orchestration system of claim 10, wherein the parameter device runs pre-stored workflow steps according to a pre-stored workflow configuration that adds mandatory and optional parameters to communications between the client system and the provider system.

12. The application program interface (API) orchestration system of claim 10, further comprising a plurality of persistence devices, one per adapter, having a power source independent from the API orchestration system.

13. The application program interface (API) orchestration system of claim 10, further comprising a plurality of persistence devices that save a current state of provider interactions and adapter state returned to the orchestration system.

14. The application program interface (API) orchestration system of claim 10, further comprising a plurality of persistence devices that save a current state of provider interactions and adapter state returned to the orchestration system, and load the adapter state into the adapters on a restart of the adapters.

15. An application program interface (API) orchestration system that facilitates communication between at least one of a plurality of client systems with at least one of a plurality of provider systems, comprising:
an orchestration module coupled to a client system, comprising:
a provider selector that receives input from the client system and associates a set of provider systems (1 to n) with the client system based upon the input received from the client system, and links the client system to a selected provider system,
an intelligent router adapted to determine eligible provider systems for each client system and to route communications between the client system to a selected eligible provider system,
a data model creator which aggregates each input from the client system and creates a canonical data model (CDM) encompassing data fragments from the client system, and
a plurality of orchestration module universal input and output interfaces, each adapted to communicate information in a predetermined standardized data format and protocol;
a plurality of adapters receiving a set of security, data format, and protocol requirements defined by an associated provider system and communicating information with the associated provider system in a manner that is compatible with the received set of security, data format, and protocol requirements, each adapter comprising:
a plurality of adapter universal input and output interfaces that are compatible with and connected to the orchestration module universal input and output interfaces of the orchestration module,
a mapper that maps data fields of the CDM and added parameters with corresponding data fields of the provider system resulting in the data format of the provider system, and
a parameter device selecting workflow steps to be executed based on data in a request adhering to the CDM.

16. The application program interface (API) orchestration system of claim 15, further comprising:
a protocol device that receives and stores protocol requirements from the provider system, and
a provider input/output device coupled to the mapper that receives provider system data in the data format of the provider system and communicates the provider system data according to the protocol requirements stored in the protocol device.

17. The application program interface (API) orchestration system of claim 16, wherein the parameter device runs pre-stored workflow steps according to a pre-stored workflow configuration that adds mandatory and optional parameters to communications between the client system and the provider system.

18. The application program interface (API) orchestration system of claim 16, further comprising a plurality of persistence devices, one per adapter, having a power source independent from the API orchestration system.

19. The application program interface (API) orchestration system of claim 16, further comprising a plurality of persistence devices that save a current state of provider interactions and adapter state returned to the orchestration system.

20. The application program interface (API) orchestration system of claim 16, further comprising a plurality of persistence devices that save a current state of provider interactions and adapter state returned to the orchestration system, and load the adapter state into the adapters on a restart of the adapters.
